(19) United States
(12) Patent Application Publication
(10) Pub. No.: US 2005/0143445 A1
(43) Pub. Date: Jun. 30, 2005

(54) Novel crystalline forms of levetiracetam

(76) Inventors: Reddy Bandi Parthasaradhi, Hyderabad (IN); Reddy Kura Rathanakar, Hyderabad (IN); Reddy Rapolu Raji, Hyderabad (IN); Reddy Dasari Muralidhara, Hyderabad (IN); Reddy Kesireddy Subash Chander, Hyderabad (IN)

Correspondence Address: CAESAR, RIVISE, BERNSTEIN,;COHEN & POKOTILOW, LTD., 11TH FLOOR, SEVEN PENN CENTER, 1635 MARKET STREET, PHILADELPHIA, PA, 19103-2212, US

(21) Appl. No.: 10/451,940

(22) PCT Filed: Mar. 18, 2003
(86) PCT No.: PCT/IN03/00058

(51) Int. Cl. A61K 31/4015; C07D 207/12

(52) U.S. Cl. 514/424; 548/550

(57) ABSTRACT

The present invention relates to novel crystalline forms of levetiracetam, to processes for their preparation and pharmaceutical compositions containing them.

### FIELD OF THE INVENTION

The present invention relates to novel crystalline forms of levetiracetam to processes for their preparation and pharmaceutical compositions containing them.

### BACKGROUND OF THE INVENTION

Levetiracetam of formula (1): 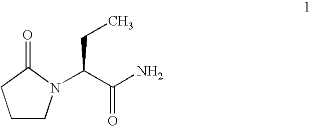
or (αS)-α-Ethyl-2-oxo-1-pyrrolidineacetamide is an anticonvulsant drug and its therapeutic uses are disclosed in U.S. Pat. No. 4,943,639.

Different synthetic methods of levetiracetam are described in U.S. Pat. No. 4,943,639, GB 2,225,322, U.S. Pat. No. 6,107,492. The known methods do not produce well define, reproducible crystalline forms.

We have discovered three novel crystalline forms of levetiracetam. The novel forms have been found to be stable over the time and reproducible. These novel forms do not automatically convert into other crystalline forms of levetiracetam.

The novel forms of levetiracetam is, thus, suitable for pharmaceutical preparations.

Thus the object of the present invention is to provide stable novel crystalline forms of levetiracetam, to provide a processes for preparation of the novel crystalline forms and to provide a pharmaceutical compositions comprising these novel crystalline forms.

### SUMMARY OF THE INVENTION

According to one aspect of the present invention, there is provided a novel crystalline Form I of levetiracetam characterized by an x-ray powder diffraction pattern having peaks expressed as 2θ at about 10.1, 15.1, 18.6, 20.4, 20.6, 22.2, 23.4, 23.9, 24.5, 26.9, 30.4, 31.0, 36.9, 45.6 degrees. FIG. 1 shows typical Form I x-ray powder diffraction pattern.

According to another aspect of the present invention, there is provided a novel crystalline Form II of levetiracetam characterized by an x-ray powder diffraction pattern having peaks expressed as 2θ at about 10.1, 14.9, 15.1, 18.5, 20.1, 20.5, 22.2, 23.3, 23.8, 24.4, 26.8, 28.9, 30.0, 30.5, 35.7, 36.3 degrees. FIG. 2 shows typical Form II x-ray powder diffraction pattern.

According to another aspect of the present invention, there is provided a novel crystalline Form III of levetiracetam characterized by an x-ray powder diffraction pattern having peaks expressed as 2θ at about 14.9, 20.6, 30.0, 30.6 degrees. FIG. 3 shows typical Form III x-ray powder diffraction pattern.

According to another aspect of the present invention there is provided a process for preparation of the Form I of levetiracetam comprising the steps of:
a) mixing levetiracetam and a suitable solvent;
b) maintaining at 15° C. to 35° C. for about 30 minutes to 4 hours;
c) isolating the Form I of levetiracetam.

The suitable solvent is selected from the group consisting of acetone, methyl isobutyl ketone, methanol, isopropyl alcohol, ethanol, butanol, acetonitrile, tetrahydrofuran, chloroform, diisopropyl ether, dioxane methyl tert-butyl ether.

According to another aspect of the present invention there is provided a process for preparation of the Form II of levetiracetam comprising the steps of:
a) dissolving levetiracetam in water;
b) leaving the solution at about 25° C. to about 30° C. till complete evaporation of water.

According to another aspect of the present invention there is provided a process for preparation of the Form III of levetiracetam comprising the steps of:
a) dissolving levetiracetam in dimethyl sulfoxide;
b) vacuum drying or spray drying;
c) washing with diisopropyl ether.
   levetiracetam prepared by any of the known methods can be used in the above processes.

According to another aspect of the present invention there is provided a pharmaceutical composition comprising Form I or Form II or Form III levetiracetam.

### BRIEF DESCRIPTION OF THE DRAWINGS

FIG. 1 is a x-ray powder diffraction pattern of Form I levetiracetam.

FIG. 2 is a x-ray powder diffraction pattern of Form II levetiracetam.

FIG. 3 is a x-ray powder diffraction pattern of Form III levetiracetam.

x-Ray powder diffraction spectrum was measured on a Siemens diffractometer.

### DETAILED DESCRIPTION OF THE INVENTION

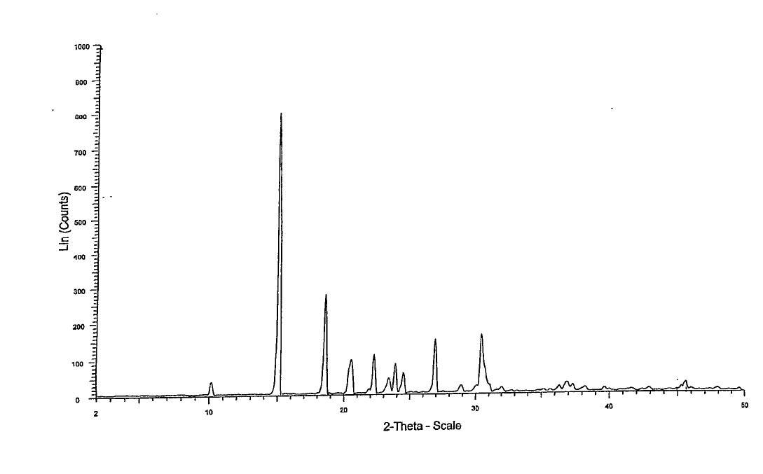

According to one aspect of the present invention, there is provided a novel crystalline Form I of levetiracetam characterized by an x-ray powder diffraction pattern having peaks expressed as 2θ at about 10.1, 15.1, 18.6, 20.4, 20.6, 22.2, 23.4, 23.9, 24.5, 26.9, 30.4, 31.0, 36.9, 45.6 degrees. FIG. 1 shows typical Form I x-ray powder diffraction pattern.

According to another aspect of the present invention, there is provided a process for preparation of the Form I of levetiracetam. Thus levetiracetam is mixed with a suitable solvent. The suitable solvent is acetone, methyl isobutyl ketone, methanol, isopropyl alcohol, ethanol, butanol, acetonitrile, tetrahydrofuran, chloroform, diisopropyl ether or dioxane methyl tert-butyl ether; or mixture thereof. Preferable solvents are acetone, ethanol and isopropyl alcohol. The contents are maintained at 15° C. to 35° C. for about 30 minutes to 4 hours. The Form I of levetiracetam is separated by filtration. The levetiracetam used in the process may be obtained by a known method. Form II or Form III of levetiracetam may also be used in the process.

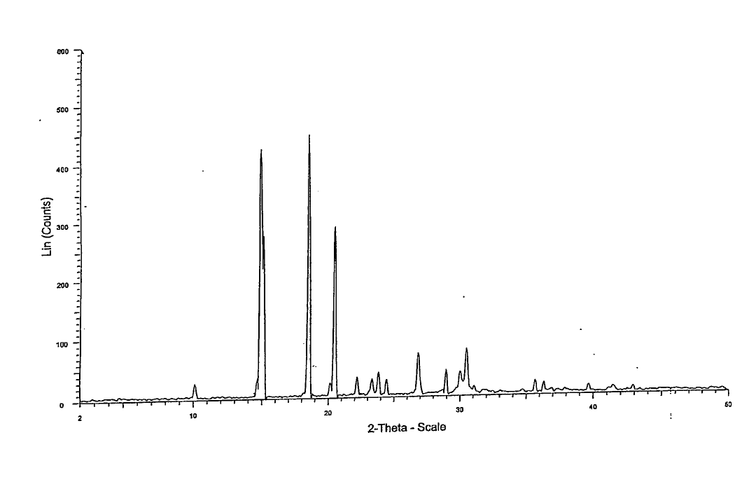

According to another aspect of the present invention, there is provided a novel crystalline Form II of levetiracetam characterized by an x-ray powder diffraction pattern having peaks expressed as 2θ at about 10.1, 14.9, 15.1, 18.5, 20.1, 20.5, 22.2, 23.3, 23.8, 24.4, 26.8, 28.9, 30.0, 30.5, 35.7, 36.3 degrees. FIG. 2 shows typical Form II x-ray powder diffraction pattern.

According to another aspect of the present invention there is provided a process for preparation of the Form II of levetiracetam. Thus levetiracetam is dissolved in water. The solvent may, if necessary, be heated to effect dissolution. Then the solution is left for complete evaporation of water at about 25° C. to about 30° C. to obtain the Form II of levetiracetam. The levetiracetam used in the process may be obtained by a known method. Form I or Form III of levetiracetam may also be used in the process.

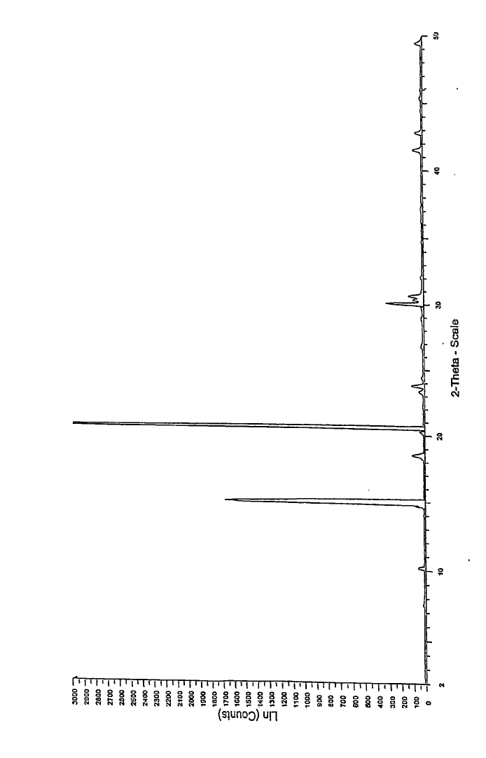

According to another aspect of the present invention, there is provided a novel crystalline Form III of levetiracetam characterized by an x-ray powder diffraction pattern having peaks expressed as 2θ at about 14.9, 20.6, 30.0, 30.6 degrees. FIG. 3 shows typical Form III x-ray powder diffraction pattern.

According to another aspect of the present invention there is provided a process for preparation of the Form III of levetiracetam. Thus levetiracetam is dissolved in dimethyl sulfoxide and the solution is subjected to vacuum drying or spray drying. The solid obtained is washed with diisopropyl ether to obtain the Form III of levetiracetam. The levetiracetam used in the process may be obtained by a known method. Form I or Form II of levetiracetam may also be used in the process.

According to another aspect of the present invention there is provided a pharmaceutical composition comprising the Form I or the Form II or the Form III of levetiracetam. The forms of levetiracetam may be formulated in a form suitable for oral administration or injection.

The following examples are given for the purpose of illustrating the present invention and should not be considered as limitations on the scope or spirit of the invention.

### EXAMPLE 1

Levetiracetam (10 gm) is mixed with acetone (50 ml) and heated to reflux. Then cooled to 25° C. to 30° C. and maintained at this temperature for 2 hours. The separated solid is filtered and dried to give 9.0 gm of Form I of levetiracetam.

### EXAMPLE 2

Levetiracetam (2 gm) is dissolved in water (10 ml) and left it for evaporation at about 25° C. for 60 hours to obtain Form II of levetiracetam in quantitative yield.

### EXAMPLE 3

Levetiracetam (1 gm) is dissolved in dimethyl sulfoxide at 25° C. The solution is subjected to vacuum dried at 62° C. under high vacuum for 4 hours. The solid obtained is washed with diisopropyl ether to give Form III of levetiracetam in quantitative yield.

### EXAMPLE 4

Levetiracetam (1 gm) is dissolved in dimethyl sulfoxide at 25° C. The solution is subjected to spray drying. The solid obtained is washed with diisopropyl ether to give Form III of levetiracetam in quantitative yield.

### EXAMPLE 5

Example 1 is repeated using Form II of levetiracetam instead of levetiracetam to give Form I of levetiracetam.

### EXAMPLE 6

Example 2 is repeated using Form III of levetiracetam instead of levetiracetam to give Form II of levetiracetam.

### EXAMPLE 7

Example 3 is repeated using Form I of levetiracetam instead of levetiracetam to give Form III of levetiracetam.

1. A crystalline Form I of levetiracetam.

2. A crystalline form of levetiracetam, characterized by an x-ray powder diffraction pattern having peaks expressed as 2θ at about 10.1, 15.1, 18.6, 20.4, 20.6, 22.2, 23.4, 23.9, 24.5, 26.9, 30.4, 31.0, 36.9, and 45.6 degrees.

3. A crystalline form of levetiracetam, characterized by an x-ray powder diffraction pattern as in FIG. 1.

4. A process for preparation of Form I levetiracetam of claim 1, comprising the steps of:
a) mixing levetiracetam and a suitable solvent to form a mixture;
b) maintaining the mixture at 15° C. to 35° C. for about 30 minutes to 4 hours; and
c) isolating the Form I of levetiracetam;
wherein suitable solvent is selected from the group consisting of acetone, methyl isobutyl ketone, methanol, isopropyl alcohol, ethanol, butanol, acetonitrile, tetrahydrofuran, chloroform, diisopropyl ether, dioxane and methyl tert-butyl ether.

5. A process according to claim 4, wherein the suitable solvent is acetone.

6. A crystalline Form II of levetiracetam.

7. A crystalline form of levetiracetam, characterized by an x-ray powder diffraction pattern having peaks expressed as 2θ at about 10.1, 14.9, 15.1, 18.5, 20.1, 20.5, 22.2, 23.3, 23.8, 24.4, 26.8, 28.9, 30.0, 30.5, 35.7, and 36.3 degrees.

8. A crystalline form of of levetiracetam, characterized by an x-ray powder diffraction pattern as in FIG. 2.

9. A process for preparation of Form II levetiracetam of claim 6, comprising the steps of:
a) dissolving levetiracetam in water; and
b) leaving the solution at about 25° C. to about 30° C. until there is complete evaporation of water.

10. A crystalline Form III of levetiracetam.

11. A crystalline form of levetiracetam, characterized by an x-ray powder diffraction pattern having peaks expressed as 2θ at about 14.9, 20.6, 30.0, and 30.6 degrees.

12. A crystalline form of levetiracetam, characterized by an x-ray powder diffraction pattern as in FIG. 3.

13. A process for preparation of Form III of levetiracetam of claim 10, comprising the steps of:
a) dissolving levetiracetam in dimethyl sulfoxide to form a solution;
b) vacuum drying or spray drying the solution of step (a); and
c) washing the product of step (b) with diisopropyl ether.

14. A process according to claim 13, wherein the solution is subjected to spray drying.

15. A process according to claim 13, wherein the solution is subjected to vacuum drying.

16. A pharmaceutical composition comprising a crystalline form of levetiracetam and a pharmaceutically acceptable carrier.

17. A pharmaceutical composition of claim 16, wherein the crystalline form of levetiracetam is the Form I levetiracetam of claim 1.

18. A pharmaceutical composition of claim 16, wherein the crystalline form of levetiracetam is the Form II levetiracetam of claim 5.

19. A pharmaceutical composition of claim 16, wherein the crystalline form of levetiracetam is the Form III of levetiracetam of claim 9.
